(19) United States
(12) Patent Application Publication
(10) Pub. No.: US 2015/0157473 A1
(43) Pub. Date: Jun. 11, 2015

(54) METHOD FOR AUTOMATICALLY ADJUSTING A DAMPING LEVEL PROVIDED BY AN ARTIFICIAL KNEE JOINT, AND THE ARTIFICIAL KNEE JOINT

(71) Applicant: Teh Lin Prosthetic & Orthopaedic Inc., New Taipei City (TW)

(72) Inventors: Hung-Jen Lai, Taipei City (TW); Ying-Ming Chung, Taipei City (TW); Jian-Liang Chen, Hualien City (TW); Chin-Wei Chen, Taipei City (TW); Chen-Hsien Chang, Keelung City (TW); Jian-Hong Lin, New Taipei City (TW); Jian-Yu Chen, (TW)

(21) Appl. No.: 14/510,079

(22) Filed: Oct. 8, 2014

(30) Foreign Application Priority Data
Dec. 11, 2013 (TW) 102145564

(51) Int. Cl. A61F 2/68; A61F 2/64

(52) U.S. Cl. CPC A61F 2/68; A61F 2/64; A61F 2002/6818; A61F 2002/764

(57) ABSTRACT

An artificial knee joint is to be connected between a prosthetic thigh and a prosthetic lower leg. The artificial knee joint includes a knee joint body, a processor mounted in the knee joint body, a damping unit that is coupled to the processor and configurable to provide various damping levels, and an accelerometer coupled to the processor. The accelerometer is configured to measure acceleration subjected to the artificial knee joint, and to generate and transmit a measuring signal according to the measurement to the processor. The processor is configured to control the damping unit to provide one of the damping levels, based on the measuring signal.

### CROSS-REFERENCE TO RELATED APPLICATION

This application claims priority of Taiwanese Application No. 102145564, filed on Dec. 11, 2013.

### FIELD OF THE INVENTION

The invention relates to an artificial knee joint and a method for automatically adjusting a damping level provided by the artificial knee joint.

### BACKGROUND OF THE INVENTION

A prosthetic leg is typically provided to a user (e.g., a leg amputee) in order to allow the user to perform normal activities such as walking. Generally, a prosthetic leg includes a prosthetic thigh, a prosthetic lower leg, and an artificial knee joint that interconnects the prosthetic thigh and the prosthetic lower leg.

A conventional artificial knee joint includes a damping unit that is configured to provide a variable damping effect (e.g., a number of different damping levels) for reducing the impact to the conventional artificial knee joint attributed to activities of the user. For example, the damping unit may include a pneumatic cylinder. Air pressure in the pneumatic cylinder may be adjustable to correspond to different damping levels. When it is desired to implement a different damping level (e.g., the user is about to start jogging), the user may operate an adjusting means to adjust the air pressure in the pneumatic cylinder.

However, the conventional artificial knee joint requires the user to manually operate the adjusting means to assign one of the damping levels, according to different activities of the user. It is desirable for a prosthetic leg to include an artificial knee joint that is capable of automatically adjusting its damping level.

### SUMMARY OF THE INVENTION

Therefore, one object of the present invention is to provide a method that addresses the aforementioned drawbacks of the prior art.

Accordingly, a method of this invention is for automatically adjusting a damping level provided by a damping unit. The damping unit is included in an artificial knee joint that connects a prosthetic thigh to a prosthetic lower leg. The artificial knee joint further includes a processor and an accelerometer coupled to the processor. The damping unit is coupled to the processor and is configurable to provide various damping levels. The method comprises the following steps of:

(a) by the accelerometer, measuring acceleration subjected to the artificial knee joint, and generating and transmitting a measuring signal according to the measurement to the processor; and

(b) controlling, by the processor, the damping unit to provide one of the damping levels based on the measuring signal.

Another object of the present invention is to provide an artificial knee joint that is configured to execute the method of the present invention.

Accordingly, an artificial knee joint of the present invention is to be connected between a prosthetic thigh and a prosthetic lower leg. The artificial knee joint comprises a knee joint body, a processor mounted in the knee joint body, a damping unit that is coupled to the processor and configurable to provide various damping levels, and an accelerometer coupled to the processor.

The accelerometer is configured to measure acceleration subjected to the artificial knee joint, and to generate and transmit a measuring signal according to the measurement to the processor. The processor is configured to control the damping unit to provide one of the damping levels based on the measuring signal.

### BRIEF DESCRIPTION OF THE DRAWINGS

Other features and advantages of the present invention will become apparent in the following detailed description of the embodiment with reference to the accompanying drawings, of which:

FIG. 1 illustrates an artificial knee joint used in a prosthetic leg according to an embodiment of the present invention;

FIG. 2 is a block diagram of the artificial knee joint;

FIG. 3 is a flowchart of a method for automatically adjusting a damping level provided by a damping unit, according to the embodiment of the present invention; and

FIG. 4 is a chart showing a weighted mean calculated over time.

### DETAILED DESCRIPTION OF THE EMBODIMENTS

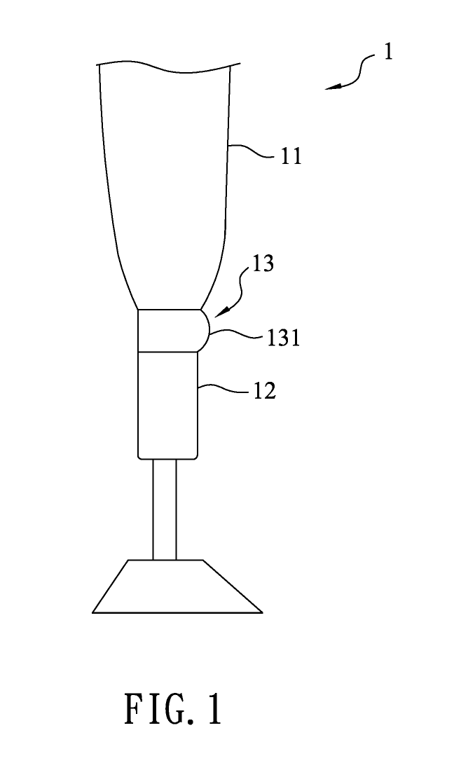

Referring to FIG. 1, an artificial knee joint 13 according to an embodiment of the present invention is used in a prosthetic leg 1 for connecting a prosthetic thigh 11 to a prosthetic lower leg 12.

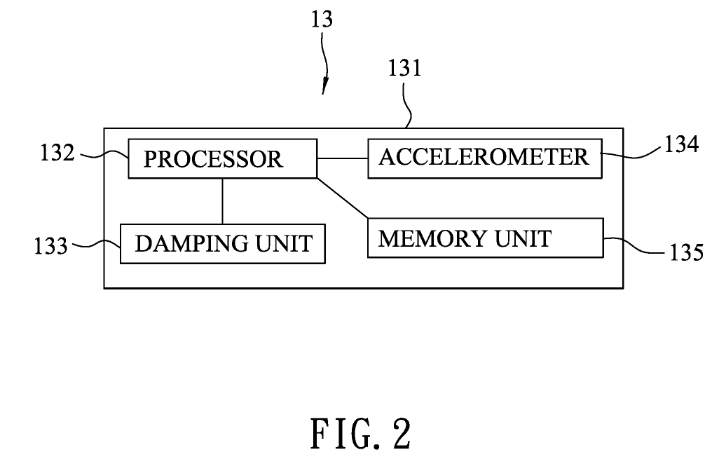

Further referring to FIG. 2, the artificial knee joint 13 includes a knee joint body 131 connected between the prosthetic thigh 11 and the prosthetic lower leg 12, a processor 132 mounted in the knee joint body 131, a damping unit 133 disposed in the knee joint body 131 and coupled to the processor 132, an accelerometer 134 disposed in the knee joint body 131 and coupled to the processor 132, and a memory unit 135.

In this embodiment, the damping unit 133 may be implemented using a pneumatic damper or a hydraulic damper, and is configurable by the processor 132 to provide various damping levels. In this embodiment, the damping unit 133 is configured to provide nine different damping levels numbered as 1 to 9, respectively, with a larger number indicating a stronger damping force. When a user wearing the prosthetic leg 1 is in motion (such as walking, running, etc.), the damping force provided by the damping unit 133 is able to reduce impacts subjected to the artificial knee joint 13. It is noted that when the impacts are more severe, a correspondingly higher damping level should be employed.

The accelerometer 134 is embodied using a three-axis accelerometer, and is configured to measure acceleration subjected to the artificial knee joint 13.

The memory unit 135 maybe embodied using a non-volatile memory, such as a flash memory, an electrically-erasable programmable read-only memory (EEPROM), etc.

The memory unit 135 stores parameters related to operations of the artificial knee joint 13, such as a plurality of damping parameters used for control of the damping unit 133, each of the damping parameters being associated respectively with a different damping level. The memory unit 135 further stores a plurality of predetermined ranges, each of the predetermined ranges corresponding respectively with the damping levels.

In this embodiment, the processor 132 is configured to calculate a weighted mean of gradient in the acceleration subjected to the artificial knee joint 13 measured by the accelerometer 134. The processor 132 then compares the weighted mean with the plurality of predetermined ranges to determine which one of the damping levels should be provided by the damping unit 133.

For example, the accelerometer 134 may measure acceleration subjected to the artificial knee joint 13 along three independent axes (e.g., an X-axis, a Y-axis and a Z-axis in a Cartesian coordinate system). Then, the accelerometer 134 may periodically generate and transmit a measuring signal according to the measurement to the processor 132. The measuring signal includes a value of the acceleration along each of the three axes. The processor 132 is configured to control the damping unit 133 to provide one of the damping levels, based on a plurality of the measuring signals received within a predetermined time period.

Specifically, the processor 132 is configured to calculate a gradient in the acceleration along each of the three axes based on the value of the acceleration, to calculate a weighted mean of the gradients (each of the gradients is given a specific weight, and each specific weight is a predetermined value stored in the memory unit 135), and to control the damping unit 133 to provide one of the damping levels based on the weighted mean.

In this embodiment, the damping unit 133 is controlled by the processor 132 to automatically provide four of the damping levels (e.g., the damping levels 2, 3, 5 and 7), and the memory unit 135 stores four different predetermined ranges each corresponding respectively to one of the damping levels listed above. Accordingly, when the weighted mean is within a first predetermined range that represents the artificial knee joint 13 operating in a slow walk state, the damping unit 133 is controlled to provide a first damping level (e.g., the damping level 2). Similarly, when the weighted mean is within a second predetermined range that is greater than the first predetermined range and that represents the artificial knee joint 13 operating in a moderate walk state, the damping unit 133 is controlled to provide a second damping level greater than the first damping level (e.g., the damping level 3) . When the weighted mean is within a third predetermined range that is greater than the second predetermined range and that represents the artificial knee joint 13 operating in a fast walk state, the damping unit 133 is controlled to provide a third damping level greater than the second damping level (e.g., the damping level 5). Furthermore, when the weighted mean goes beyond the third predetermined range, the damping unit 133 is controlled to provide a fourth damping level greater than the third damping level (e.g. , the damping level 7) . It is noted that in other embodiments, different damping levels maybe utilized in each of the above situations, based on the user's preference.

In use, when a user of the prosthetic leg 1 starts walking in a slow pace, the weighted mean associated with the acceleration that is subjected to the artificial knee joint 13 is within the first predetermined range. The processor 132 in turn controls the damping unit 133 to provide the first damping level (i.e., the damping level 2).

Then, when the user changes his/her activity (e.g., starts jogging or walking at a faster pace), the weighted mean associated with the acceleration that is subjected to the artificial knee joint 13 is within or beyond the third predetermined range. As a result, the processor 132 in turn controls the damping unit 133 to provide the third or fourth damping level (i.e., the damping level 5 or 7).

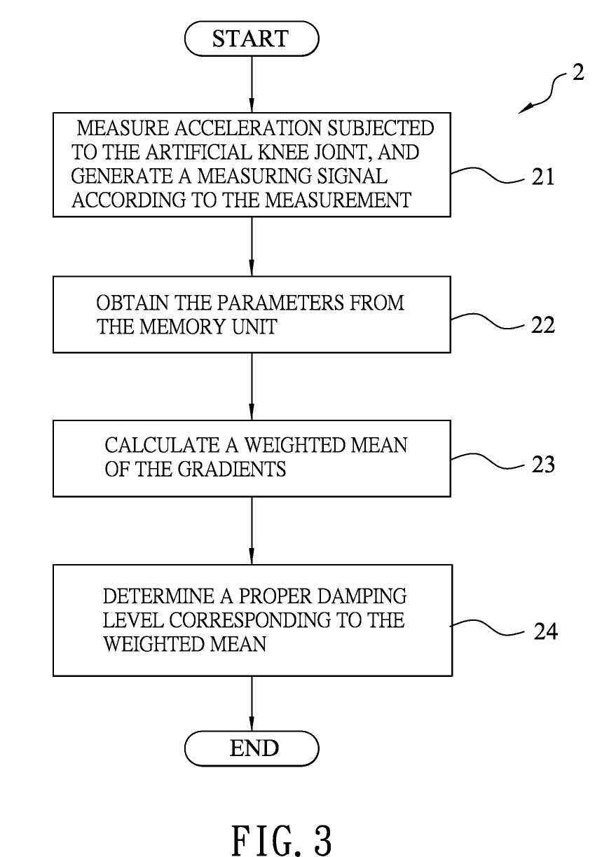

Referring to FIG. 3, a method for automatically adjusting a damping level provided by the damping unit 133 of the artificial knee joint 13 according to an embodiment of the present invention will now be described.

When a user of the prosthetic leg 1 starts taking steps, in step 21, the accelerometer 134 continuously measures acceleration subjected to the artificial knee joint 13 along three independent axes (in this embodiment, the axes are an X-axis, a Y-axis and a Z-axis in a Cartesian coordinate system) . Then, the accelerometer 134 periodically generates a measuring signal according to the measurement, and transmits the measuring signal to the processor 132. The measuring signal generated by the accelerometer includes a value of the acceleration along each of the three axes.

In step 22, the processor 132 obtains the parameters from the memory unit 135 for processing the measuring signal.

In step 23, the processor 132 calculates the gradient in the acceleration along each of the three axes based on the value of the acceleration, and calculates a weighted mean of the gradients based on the specific weights. For example, the weighted mean may be calculated using the following equation:

Weighted mean=Δₓ*Wₓ+Δ_{y}*W_{y}+Δ_{z}*W_{z}

where Δₓ, Δ_{y} and Δₓ represent the gradients in the acceleration along the X-axis, the Y-axis and the Z-axis, respectively, and Wₓ, W_{y} and W_{z} represent the specific weights given to the gradients Δₓ, Δ_{y} and Δ_{z}, respectively. In this embodiment, Wₓ=0.33, W_{y}=0.33, and W_{z}=0.33.

In step 24, the processor 132 determines a proper damping level corresponding to the weighted mean calculated above, and controls the damping unit 133 to provide the corresponding damping levels.

For example, the following Table 1 shows an exemplary set of parameters obtained from the memory unit 135, the processor 132 using the parameters to determines the proper damping level.

TABLE 1

| Predetermined | | |
|---|---|---|
| Range for the | | |
| Weighted Mean | Corresponding | Represented |
| (m/s³) | Damping Level | Activity State |
| 11-105 | First (damping | Slow Walk |
| | level 2) | |
| 106-170 | Second (damping | Moderate Walk |
| | level 3) | |
| 171-275 | Third (damping | Fast Walk |
| | level 5) | |
| >276 | Fourth (damping | High Intensity |
| | level 7) | |

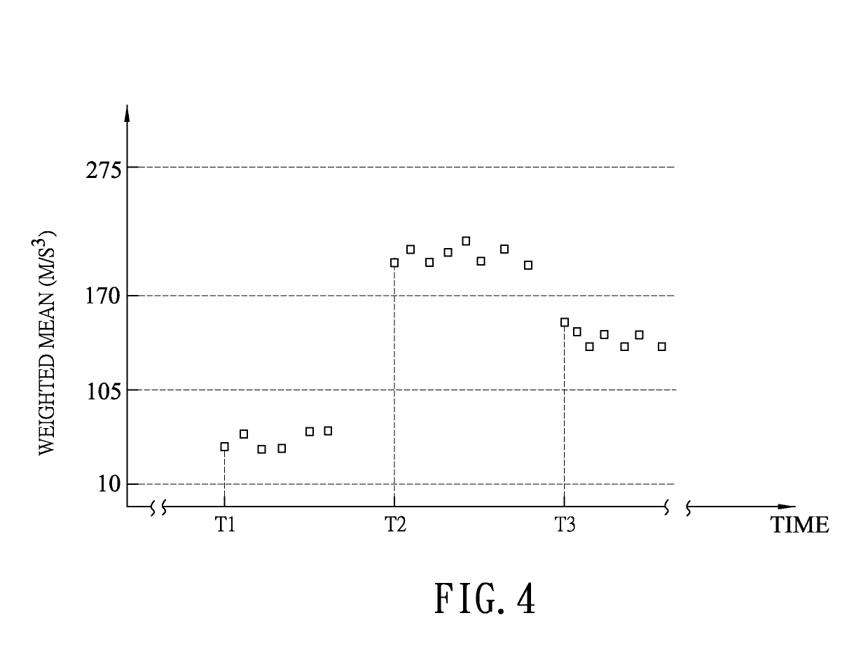

FIG. 4 lists calculated weighted mean associated with the prosthetic leg 1 over time. In a first instant (T₁), the user of the prosthetic leg 1 begins a slow walk, and the weighted mean calculated thereafter indicates the damping unit 133 to provide the first damping level (i.e., the damping level 2). In a second instant (T₂), the user of the prosthetic leg 1 starts walking faster, and the weighted mean calculated thereafter indicates the damping unit 133 to provide the second damping level (i.e., the damping level 3). In a third instant (T₃), the user of the prosthetic leg 1 slows down slightly, and the weighted mean calculated thereafter indicates the damping unit 133 to provide the second damping level.

In some embodiments, when the calculated weighted mean is lower than 10 (m/s³), the processor 132 may control the damping unit 133 to provide the first damping level. This may reduce the power consumption of the artificial knee joint 13 attributed to the damping unit 133 changing the provided damping level.

In some embodiments, the artificial knee joint 13 may further include an interface unit (not shown in the drawings) for allowing a user to adjust the parameters stored in the memory unit 135. The interface unit may include a wireless transmission component such as an InfraRed transmitter or a Bluetooth transmitter . The user may operate a remote device to communicate with the interface unit and adjust the parameters stored in the memory unit 135 via the interface unit. In some embodiments, the user may execute an application to adjust the parameters, and transmit the adjusted parameters to the artificial knee joint 13 via the interface unit.

To sum up, the artificial knee joint 13 of the present invention employs the accelerometer 134 that measures acceleration along each of the three axes, and the processor 132 is configured to determine a proper damping level for a current activity state of the artificial knee joint 13, and to control the damping unit 133 to provide the proper damping level. As a result, the artificial knee joint 13 executing the method of the present invention is able to achieve the effect of automatically adjusting the damping level.

While the present invention has been described in connection with what is considered the most practical embodiment, it is understood that this invention is not limited to the disclosed embodiments but is intended to cover various arrangements included within the spirit and scope of the broadest interpretation so as to encompass all such modifications and equivalent arrangements.

What is claimed is:

1. An artificial knee joint configured to connect a prosthetic thigh to a prosthetic lower leg, said artificial knee joint comprising:
a knee joint body;
a processor mounted in said knee joint body;
a damping unit disposed in said knee joint body, coupled to said processor, and configurable to provide various damping levels; and
an accelerometer disposed in said knee joint body, and coupled to said processor, said accelerometer being configured to measure acceleration subjected to said artificial knee joint, and to generate and transmit a measuring signal according to the measurement to said processor,
wherein said processor is configured to control said damping unit to provide one of the damping levels based on the measuring signal.

2. The artificial knee joint of claim 1, wherein said accelerometer is configured to periodically generate and transmit one measuring signal to said processor, and said processor controls the damping unit based on a plurality of the measuring signals received within a predetermined time period.

3. The artificial knee joint of claim 2, wherein:
said accelerometer is a three-axis accelerometer, and is configured to measure acceleration subjected to said artificial knee joint along three independent axes, each of the measuring signals generated by said accelerometer including a value of the acceleration along each of the three independent axes; and
said processor is configured to calculate a gradient in the acceleration along each of the three independent axes based on the value of the acceleration, and to control said damping unit based on the gradient in the acceleration along each of the three independent axes.

4. The artificial knee joint of claim 3, wherein said processor is configured to:
calculate the gradients in the acceleration respectively along the three independent axes;
calculate a weighted mean of the gradients, each of which is given a specific weight; and
control said damping unit to provide one of the damping levels based on the weighted mean.

5. The artificial knee joint of claim 4, wherein each of the specific weights is a predetermined value.

6. The artificial knee joint of claim 4, wherein said processor controls said damping unit to provide:
a first damping level, when the weighted mean is within a first predetermined range that represents the artificial knee joint operating in a slow walk state;
a second damping level greater than the first damping level, when the weighted mean is within a second predetermined range that is greater than the first predetermined range and that represents the artificial knee joint operating in a moderate walk state;
a third damping level greater than the second damping level, when the weighted mean is within a third predetermined range that is greater than the second predetermined range and that represents the artificial knee joint operating in a fast walk state; and
a fourth damping level greater than the third damping level, when the weighted mean goes beyond the third predetermined range.

7. A method for automatically adjusting a damping level provided by a damping unit included in an artificial knee joint that connects a prosthetic thigh to a prosthetic lower leg, the artificial knee joint further including a processor and an accelerometer coupled to the processor, the damping unit being coupled to the processor and configurable to provide various damping levels, said method comprising the following steps of:
(a) by the accelerometer, measuring acceleration subjected to the artificial knee joint, and generating and transmitting a measuring signal according to the measurement to the processor; and
(b) controlling, by the processor, the damping unit to provide one of the damping levels based on the measuring signal.

8. The method of claim 7, wherein, in step (a), the accelerometer periodically generates and transmits one measuring signal to the processor, and in step (b), the processor controls the damping unit based on a plurality of the measuring signals received within a predetermined time period.

9. The method of claim 8, the accelerometer being a three-axis accelerometer, wherein:
in step (a), the accelerometer measures acceleration subjected to the artificial knee joint along three independent axes, and each of the measuring signals generated by the accelerometer includes a value of the acceleration along each of the three independent axes;
in step (b), the processor calculates a gradient in the acceleration along each of the three independent axes based on the value of the acceleration, and controls the damping unit based on the gradient in the acceleration along each of the three independent axes.

10. The method of claim 9, wherein step (b) includes the following sub-steps of:
calculating the gradients in the acceleration respectively along the three independent axes;
calculating a weighted mean of the gradients, each of which is given a specific weight; and
controlling the damping unit to provide one of the damping levels based on the weighted mean.

11. The method of claim 10, wherein each of the specific weights is a predetermined value.

12. The method of claim 10, wherein, instep (b), the processor controls the damping unit to provide:
a first damping level, when the weighted mean is within a first predetermined range that represents the artificial knee joint operating in a slow walk state;
a second damping level greater than the first damping level, when the weighted mean is within a second predetermined range that is greater than the first predetermined range and that represents the artificial knee joint operating in a moderate walk state;
a third damping level greater than the second damping level, when the weighted mean is within a third predetermined range that is greater than the second predetermined range and that represents the artificial knee joint operating in a fast walk state; and
a fourth damping level greater than the third damping level, when the weighted mean goes beyond the third predetermined range.
